(19) United States
(12) Patent Application Publication
(10) Pub. No.: US 2013/0041178 A1
(43) Pub. Date: Feb. 14, 2013

(54) METHOD FOR PREPARING OPTICALLY ACTIVE N-METHYLAMINO ACIDS AND OPTICALLY ACTIVE N-METHYLAMINO ACID AMIDES

(71) Applicants: Sachiko Arie, Matsudo-shi (JP); Masaki Sugita, Niigata-shi (JP)

(72) Inventors: Sachiko Arie, Matsudo-shi (JP); Masaki Sugita, Niigata-shi (JP)

(73) Assignee: MITSUBISHI GAS CHEMICAL COMPANY, INC., Tokyo (JP)

(21) Appl. No.: 13/636,865

(22) PCT Filed: Mar. 15, 2011
(86) PCT No.: PCT/JP2011/056046

§ 371 Date: Nov. 2, 2012

(30) Foreign Application Priority Data
Mar. 23, 2010 (JP) 2010-066157

(51) Int. Cl. C07C 227/18; C07C 231/12

(52) U.S. Cl. 562/575; 564/198

(57) ABSTRACT

The present invention relates to a method for preparing optically active N-methylamino acids and/or optically active N-methylamino acid amides by subjecting the cells or treatment products of a microorganism belonging to the Mycoplana genus to the hydrolysis with N-methylamino acid amide to give an optically active N-methylamino acid and an optically active N-methylamino acid amide. According to the present invention, optically active N-methylamino acids and optically active N-methylamino acid amides useful as the raw material of pharmaceuticals can be stereoselectively obtained with good efficiency.

### CROSS-REFERENCE TO RELATED APPLICATION

This application is based upon and claims the benefit of priority from the prior Japanese Patent Application No. 2010-066157 filed on Mar. 23, 2010, the entire contents of which are incorporated herein by reference.

### TECHNICAL FIELD

The present invention relates to a process for preparing optically active N-methylamino acids and optically active N-methylamino acid amides.

### BACKGROUND ART

Optically active N-methylamino acids and optically active N-methylamino acid amides are used extensively as the raw materials of peptide drugs, specifically used as the raw materials of antirheumatic drugs and anticancer drugs (Japanese Patent Laid-Open Publication No. 2001-518515 (WO1999/17792), WO1999/17792, Japanese Patent Laid-Open Publication No. 2001-514659 (WO1998/40092), and Japanese Patent Laid-Open Publication No. 2000-502092 (WO1997/22621)).

A number of pharmaceuticals containing an amino acid as a raw material have been developed, but peptide pharmaceuticals have a disadvantage of incapable of oral administration due to their easy decomposition by lyases in the digestive system. On the other hand, peptide pharmaceuticals containing non-natural amino acids or chemically modified amino acids as the parts of their structures are resistant to lyases and thus capable of oral administration. It is thus predicated that the optically active N-methylamino acids and the optically active N-methylamino acid amides are very useful as the raw materials of pharmaceuticals.

The optically active N-methylamino acids can be synthesized by the reaction of an optically active amino acid with dimethyl sulfate or methyl iodide (Can. J. Chem., 55(5), 916 (1977)). However, it is difficult to obtain a monomethyl product alone, and the reaction may often proceed to dimethylation. Therefore, it was difficult to prepare efficiently the optically active N-methylamino acids.

It has been disclosed the alternative method for industrially preparing the optically active N-methylamino acids by protecting preliminarily the amino group of the optically active amino acid with a Boc group, a Cbz group or a Fmoc group before methylation (U.S. Pat. No. 5,587,506, U.S. Pat. No. 6,218,572). However, an expensive protecting agent is required for the method for protecting the amino group and additionally deprotection is required after methylation, which leads to the complicated steps. Moreover, the methylating agent must be used in an excessive amount, so that the above described method cannot be said advantageous from the economic viewpoint.

Thus, a method for utilizing enzymes present in microorganisms is proposed as a method for inexpensively preparing an optically active N-methylamino acid. The microorganisms having enzymes which can produce natural or non-natural amino acids include the ones such as a gene recombinant Escherichia coli strain pMCA1/JM109 (FERM BP-10334) having a gene extracted from Xanthobacter Flavus which may hydrolyze stereoselectively valinamide (WO2006/008872 (European Patent Publication, EP1770166A)). However, only two cases have hitherto been described as the biochemical methods which can be applied to the synthesis of optically active N-methylamino acids.

By way of example, it has been described in Japanese Patent Laid-Open Publication No. 2001-190298 that optically active N-methylamino acids can be produced by reacting an α-keto acid compound with an inexpensive methylamine in the presence of a transaminase. However, it is generally difficult to industrially obtain such α-keto acid compound, and thus the above described method cannot be always said to have an economic advantage. Furthermore, optically active N-methylamino acids containing a phenylalanine skeleton have merely been exemplified in the specification, and the substrate to which the transaminase can be applied is limited.

Moreover, a transaminase has also been described in FEBS J., 272, 1117 (2005) which can react methylamine with phenylpyruvic acid to prepare N-methyl phenylalanine. However, this enzyme exhibits no activities on α-ketoisovaleric acid or α-keto-β-methylvaleric acid. Thus, this transaminase cannot be used for the preparation of optically active lower aliphatic N-methylamino acids because of its very high substrate specificity and limited substrates.

In addition, there has also been described a method for stereoselectively hydrolyzing N-methylamino acid amide by utilizing an amidase present in Rhodococcus sp. AJ270 (Tetrahedron: Asymmetry, 16, 2409 (2005)). Optically active N-methylamino acid as the product by hydrolysis and the optically active N-methylamino acid amide as the unreacted material can be obtained by this method. However, the method, while having high stereoselectivities to N-methylphenylglycine amides or N-methylcyclohexylglycine amides, has low stereoselectivities to the N-methylamino acid amide. For instance, in the case of N-methylvaline amide, the optical purity of N-methylvaline obtained remains at the level of 13% ee, and thus it is difficult to use the N-methylvaline in the industrial level. Also, in the case of N-methylphenylglycine amides, the enzymatic activity is substantially decreased due to merely the difference of a substituent position on the phenyl ring, and thus it is considered that the enzyme tends to easily receive the electronic and steric influences of substrates.

Furthermore, microorganisms of Mycoplana or Mycobacterium genus which have a stereoselective hydrolysis activity on an amide bond are known (Japanese Patent Laid-Open Publication No. 2009-278914). However, the reaction with which these microorganisms are involved is the one having an organic amino acid amide which is sparingly soluble in water as a substrate, and the solvent or the reaction is also an aqueous solution of an organic solvent. On the other hand, N-methylamino acid amide is a water-soluble substrate, which is desirably subjected to the reaction in an aqueous solution. In general, an enzyme exhibits different reaction activities depending on the properties of solvent or substrates, and thus it has been considered that the microorganisms described above have no activities on the N-methylamino acid amide.

Therefore, no methods for preparing optically active N-methylamino acids and/or optically active N-methylamino acid amides with use of an enzyme having a high activity on N-methylamino acid amide have hitherto been described. Also in the case of using such a biochemical method with use of an enzyme contained in a microorganism as described above, there are only a limited number of the methods for efficiently preparing optically active N-methylamino acids and optically active N-methylamino acid amides. Particularly, the method for preparing optically, active N-methylamino acids and optically active N-methylamino acid amides having lower alkyl groups have not yet established as far as the present inventors know.

### SUMMARY OF THE INVENTION

The present inventors have now found that a microorganism having a stereoselective activity of hydrolyzing N-methylamino acid amide is present among the existing microorganisms. The microorganism was found to have an extremely high stereoselectivity also in N-methylamino acid amides having a lower alkyl group. Optically active N-methylamino acids and optically active N-methylamino acid amides have successfully been prepared by stereoselectively hydrolyzing the N-methylamino acid amides having a lower alkyl group with good efficiencies by subjecting the cells or treatment products of this microorganism to reaction. The present inventors have hereby succeeded in establishing a novel method for efficiently preparing the optically active N-methylamino acids and optically active N-methylamino acid amides with use of the biocatalyst. The present invention is based on these findings.

Consequently, the object of the present invention is to provide a method for efficiently preparing optically active N-methylamino acids and/or optically active N-methylamino acid amides which are useful as the raw materials of pharmaceuticals by utilizing a biocatalyst.

That is, the present invention relates to a method for efficiently preparing optically active N-methylamino acids and/or optically active N-methylamino acid amides from N-methylamino acid amide as shown in the following (i) to (iv).

(i) A method for preparing optically active N-methylamino acids and/or optically active N-methylamino acid amides, which comprises subjecting the cells or treatments product of a microorganism belonging to the Mycoplana genus to the reaction with N-methylamino acid amide of formula (1) to give an optically active N-methylamino acid of formula (2) and an optically active N-methylamino acid amide of formula (3) as the enantiomer.

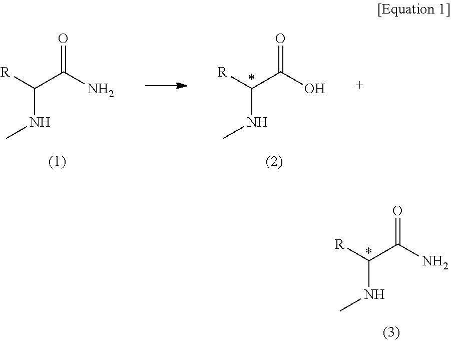

wherein R in formulae (1), (2) and (3) represents a linear or branched C1 to C4 alkyl group.

(ii) The method according to (i) described above, which further comprises:

depositing an optically active amino acid as the crystalline product from the hydrolysis mixture which contains optically active N-methylamino acids and optically active N-methylamino acid amides obtained by subjecting the cells or treatment products of a microorganism belonging to the Mycoplana genus to an N-methylamino acid amide of formula (1) and

separating the optically active N-methylamino acid and the optically active N-methylamino acid amide.

(iii) The method according to (i) or (ii) described above, wherein the microorganisms belonging to Mycoplana genus is Mycoplana ramosa or Mycoplana dimorpha.

(iv) The method according to any one of (i) to (iii) described above, wherein R in formulae (1), (2) and (3) represents an isopropyl group.

The cells or treatment products of the microorganisms employed in the present invention have the biocatalyst activity which hydrolyzes the amide bond of the N-methylamino acid amide represented by formula (1) with a high reaction rate and stereoselectivity. That is, the present invention has been accomplished by finding and focusing on a novel activity in the existing microorganisms. According to the present invention, the aimed optically active N-methylamino acid and optically active N-methylamino acid amide can be efficiently produced from the N-methylamino acid amide as the raw material. Consequently, it will be possible to produce economically (at a low cost) and supply the optically active N-methylamino acid and optically active N-methylamino acid amide which are important as the raw materials of pharmaceuticals.

### DETAILED DESCRIPTION OF THE INVENTION

The method for preparing the optically active N-methylamino acid and/or optically active N-methylamino acid amide according to the present invention comprises subjecting the cells or treatment products of a microorganism belonging to the Mycoplana genus to the reaction with N-methylamino acid amide of formula (1) to give an optically active N-methylamino acid of formula (2) and an optically active N-methylamino acid amide of formula (3) as the enantiomer. The representation “subjecting” the cells or treatment products “to the reaction” with N-methylamino acid amide of formula (1) herein means subjecting the cells or treatment products to coexistence with the N-methylamino acid amide in an aqueous solution and to reaction by its catalytic effect.

Furthermore, the phraseology “and/or” is herein used, for example, to include in the case of the optically active N-methylamino acid and/or the optically active N-methylamino acid amide, “the optically active N-methylamino acid”, “the optically active N-methylamino acid amide”, or “the optically active N-methylamino acid and the optically active N-methylamino acid amide”.

The N-methylamino acid amide as the substrate of the raw material of the present invention is an N-methylamino acid amide having a C1 to C4 linear or branched alkyl group in the side chain R of formula (1). In this connection, R preferably represents a C3 to C4 branched alkyl group. The embodiments of the group R include methyl, ethyl, n-propyl, isopropyl, n-butyl, sec-butyl and tert-butyl.

Subsequently, the specific examples of the N-methylamino acid amide of formula (1) include N-methylalanine amide, N-methylaminobutyric acid amide, N-methylnorvaline amide, N-methylvaline amide, N-methylnorleucine amide, N-methylleucine amide, N-methylisoleucine amide and N-methyl-tert-leucine amide, preferably, N-methylvaline amide, N-methylleucine amide, N-methylisoleucine amide and N-methyl-tert-leucine amide. Among them, N-methylvaline amide is particularly the appropriate substrate.

In the present invention, the N-methylamino acid amide used in the reaction may also be in the form of a mineral acid salt such as hydrochloric acid, sulfuric acid or nitric acid or an organic acid salt such as acetic acid or the like.

The microorganism or its treatment products of the present invention having an activity of stereoselectively hydrolyzing the N-methylamino acid amide of formula (1) is the one which has an activity of stereoselectively hydrolyzing the amide bond of the N-methylamino acid amide. The microorganisms having the biocatalyst activity are the organisms belonging to the Mycoplana genus and specifically include Mycoplana ramosa and Mycoplana dimorpha as the preferable ones.

Hitherto, when the cells or treatment products of the microorganisms considered to have an amino acid amide hydrolysis activity was used as a biocatalyst and including, for example, Xanthobacter genus, Protaminobacter genus, Mycobacterium genus, Pseudomonas genus, Rhodococcus genus, Serratia genus and Chromobacterium genus, the cells or treatment products of the microorganisms had little hydrolysis activity to N-methylamino acid amides.

In such situations, the present inventors have now found unexpectedly that among the existing microorganisms, that is, the ones available to persons skilled in the art such as the commercially available ones or the ones distributed by depositors, the microorganisms belonging to the Mycoplana genus have a high biocatalyst activity to N-methylamino acid amides. The microorganisms utilized in the present invention include without any limitations the ones belonging to the Mycoplana genus, which can be appropriately selected and used focusing on the desired catalyst activity.

In the present invention, the preferable specific examples of the microorganisms belonging to the Mycoplana genus include Mycoplana ramosa ATCC49678, Mycoplana dimorpha ATCC4279 and Mycoplana dimorpha NCIB9439T. In the case of using the cells or treatment products of these microorganisms as a biocatalyst, higher catalyst activity is obtained for the N-methylamino acid amide as the substrate.

In this connection, the microorganisms specifically described above are extensively employed in various research organizations and the like, and, if necessary, it is possible to directly purchase or receive a lot according to the accession number from ATCC (USA) and NCIMB (or NCIB) (UK). Furthermore, if necessary, the microorganism may be easily obtained from its actual owner by permission of the person concerned on the basis of the prescribed accession number and/or the name of the microorganism. In addition, if necessary, the other microorganisms specifically described in the specification may be easily obtained as well.

Moreover, variants or cell fusion strains derived from these microorganisms by artificial mutational means, or recombinant strains derived from these microorganisms by genetic methods, which have a capability (activity) described above may also be used in the present invention.

The culture of these microorganisms is carried out in a culture medium to which assimilable carbon sources, nitrogen sources, inorganic salts essential to the respective microorganisms, nutrients and the like have been added. Particularly, when urea is added as the nitrogen source, the microorganisms obtained by culture preferably exhibit a high activity as the biocatalyst. The microorganism shows the pH of 4 to 10, and the temperature of 20 to 50° C. during culture. Culture is carried out aerobically for about 1 day to 1 week. The microorganism is used for the reaction in the form of the cells or treatment products thereof such as culture fluids, isolated cells, cell homogenates, and further purified biocatalysts. It may be also used as a biocatalyst by immobilizing the cells or treatment products thereof according to the conventional methods.

The used amount of the cells or treatment products of the microorganisms to the N-methylamino acid amide represented by formula (1) is preferably added in a weight ratio of 0.0001 to 3 times in terms of the to the weight of the dry cells, more preferably 0.001 to 1 times. If the weight ratio is less than 0.0001 times, a long treatment time may be required due to the low reaction rate. If the weight ratio is more than 3 times, it happens that the microorganism is not preferred in terms of the utilization efficiency as the biocatalyst in spite of the shorter reaction time, and laborious treatments are required for the separation of the cells or treatment products thereof thus resulting in industrial disadvantage.

The cells or treatment products of a microorganism belonging to the Mycoplana genus are subjected to the reaction with the N-methylamino acid amide represented by formula (1) preferably at a temperature in the range of 10 to 60° C. The reaction temperature is more preferably in the range of 30 to 50° C. If the reaction temperature is lower than 10° C., treatment may require a longer time due to the slow reaction rate. On the other hand, if the reaction temperature exceeds 60° C., the biocatalyst activities of the cells or the treatment product of the cells are lowered due to their deactivation, which also accompanies the non-enzymatic decomposition of the amino acid amide thus resulting in the decrease of the optical purity of the product. Furthermore, a large amount of energy will be required for the temperature rising or cooling of the reaction fluid between reaction steps.

The reaction is preferably carried out at pH 6 to 10, more preferably pH 7 to 8. In order to adjust the pH of the reaction fluid to a suitable range, acids or bases may be appropriately added to the reaction fluid. By way of example, if the N-methylamino acid amide is used as a raw material, acids such as hydrochloric acid, sulfuric acid or nitric acid may be added for adjusting pH. Furthermore, if an acid salt of the N-methylamino acid amide is used as a raw material, the pH may be adjusted by adding bases such as sodium hydroxide, potassium hydroxide or the like. If the pH is lower than 6, the catalyst activity of the cells or the treatment products of the cells may be decreased to lead to the stagnation of the reaction. On the other hand, if the pH exceeds 10, the optical purity may be lowered notwithstanding the increase of the apparent reaction rate due to the non-enzymatic hydrolysis reaction by the bases.

Next, the cells or treatment products of the microorganism are removed from the fluid after reaction with a centrifuge or an ultrafiltration membrane. Subsequently, the optically active N-methylamino acid as the reaction product can be recovered by the extraction operation into a solvent for dissolving the optically active N-methylamino acid. When the acid or base is added for adjusting the pH described above, the neutralization treatment of the acid or base added for efficiently extracting the optically active N-methylamino acid may be carried out to form a salt insoluble into the solvent for dissolving the optically active N-methylamino acid. The organic solvent used in this case is the one which dissolves the optically active N-methylamino acid and includes, without specific limitations, for example, alcohols such as methanol, ethanol, isopropanol or isobutanol.

In addition, the organic solvent with which the optically active N-methylamino acid was extracted contains also the unreacted N-methylamino acid amide, which can be separated by a method for utilizing the difference of solubilities of the optically active N-methylamino acid and the N-methylamino acid amide. The solvent used in this case may be the one which has a low solubility to the optically active N-methylamino acid and a high solubility to the N-methylamino acid amide, and includes, for example, ketones such as acetone or methylethylketone or hydrocarbons such as toluene or xylene. The organic solvent solution from which the optically active N-methylamino acid was extracted, or the organic solvent solution described above from which the organic solvent was removed by distillation was diluted with the solvent described above for the solid-liquid separation operation such as filtration or centrifugation to give the optically active N-methylamino acid.

Then, the unreacted N-methylamino acid amide having a high optical purity can be obtained by removing the organic solvent by distillation from the organic solvent solution obtained by the solid-liquid separation operation described above. The acid salt of the optically active N-methylamino acid amide can be obtained by adding a mineral acid such as hydrochloric acid or sulfuric acid or an organic acid such as acetic acid to the organic solvent solution obtained by the solid-liquid separation operation described above. If the acid salt of the optically active N-methylamino acid amide is deposited from the organic solvent, it is recovered by conducting the solid-liquid separation operations such as filtration or centrifugation. Furthermore, the optically active N-methylamino acid amide can also be hydrolyzed with hydrochloric acid or the like to lead to the optically active N-methylamino acids.

According to the method of the present invention, for example, N-methyl-L-valine and N-methyl-D-valine amide useful as the raw materials of pharmaceuticals can be prepared from the N-methylvaline amide.

### EXAMPLES

The present invention is more specifically described with Examples and Comparative Examples, but it is not limited to these examples.

### Measurements:

The amounts or optical purities of the N-methylamino acid amide as a substrate and the optically active N-methylamino acid as the reaction product are measured under the following HPLC conditions.

### (1) Measurement of Reaction Rate

Column: Lichrosorb RP-18 (4.6φ×250 mm)

Column temperature: 30° C., Detection: R1

Eluent: 50 mM perchloric acid aqueous solution,

Flow rate: 0.5 ml/min

### (2) Measurement of Optical Purity

Column: Sumichiral OA-5000 (4.6φ×50 mm)

Column temperature: 30° C., Detection: Absorption at 260 nm

Eluent: 1 mM copper sulfate aqueous solution,

Flow rate: 1.0 ml/min

### Reference Example 1

Culture of Mycoplana ramosa ATCC49678

Mycoplana ramosa ATCC49678 which was found to stereoselectively hydrolyze the N-methylamino acid amide as a substrate with good efficiency was inoculated in a culture medium having a composition shown in Table 1 and subjected to shake culture at 30° C. for 69 hours. The culture fluid thus obtained was centrifuged at 5° C. at 1200×g of a centrifugal acceleration for 15 minutes. Supernatant was removed to give 2.65 g of a concentrated cell fluid.

TABLE 1
Composition of culture medium (the following
composition was adjusted to pH 7.0 with a 20%
sodium hydroxide aqueous solution before use)

| | |
|---|---|
| RAW MATERIAL OF CULTURE MEDIUM | USED AMOUNT |

| | | |
|---|---|---|
| Glucose | 10 | g |
| Urea | 5 | g |
| Yeast extract | 5 | g |
| Potassium dihydrogen phosphate | 1 | g |
| Magnesium sulfate heptahydrate | 0.04 | g |
| Manganese (II) chloride tetrahydrate | 1 | mg |
| Iron (II) sulfate heptahydrate | 1 | mg |
| Water | 1000 | ml |

### Example 1

### Hydrolysis of N-Methylvaline Amide

A 100 g amount of a substrate solution was prepared by dissolving 1.0 g of racemic N-methylvaline amide hydrochloride in 50 g of water in a 200 ml flask and further adjusting the solution to pH 8.0 with 20% sodium hydroxide aqueous solution before dilution with water. To each of the substrate solutions was added 0.5 g of the concentrated cell fluid of Mycoplana ramosa ATCC49678 obtained in Reference Example 1, and the mixture was subjected to reaction under stirring with a magnetic stirrer at 35° C. for 1 hour. It was observed in the HPLC measurement of the reaction fluid that 47.3% of N-methylvaline amide had been hydrolyzed. Furthermore, N-methylvaline produced by the hydrolysis was an L-form and exhibited the optical purity of 99% ee or more.

### Reference Example 2

### Culture of the Other Strains

In the same manner as Mycoplana ramosa ATCC49678 of Reference Example 1, each of the strains Mycoplana dimorpha ATCC4279 and Mycoplana dimorpha NCIB9439T selected by screening was inoculated in the culture medium having the composition shown in Table 1, and cultured under stirring at 30° C. 70 hours. The culture fluid thus obtained was centrifuged to obtain a concentrated cell fluid.

The results were shown in Table 2. In this connection, the acquisition amount of the concentrated cells in the table means the amount (g) of the concentrated cell fluid.

TABLE 2

| | ACQUISITION AMOUNT OF |
|---|---|
| STRAIN | THE CONCENTRATED CELLS |
| Mycoplane dimorpha ATCC4279 | 1.00 g |
| Mycoplane dimorpha NCIB9439T | 1.40 g |

### Example 2

### Hydrolysis of N-Methylvaline Amide

A 100 g amount of a substrate solution was prepared by dissolving 1.0 g of racemic N-methylvaline amide hydrochloride in 50 g of water in a 200 ml flask and further adjusting the solution to pH 8.0 with 20% sodium hydroxide aqueous solution before dilution with water. To each of the substrate solutions was added 0.5 g of the each concentrated cell fluid obtained in Reference Example 2, and the mixture was subjected to reaction under stirring with a magnetic stirrer at 35° C. for 1 hour. It was observed in the HPLC measurement of the reaction fluid that hydrolysis of N-methylvaline amide had advanced. Furthermore, N-methylvaline produced by the hydrolysis was an L-form in each of the cases and exhibited the optical purity of 99% ee or more.

The results are shown in Table 3. In this connection, the yield in the table means the ratio of L-N-methylvaline converted by the hydrolysis of L-N-methylvaline amide contained in the N-methylvaline amide hydrochloride as the substrate used in the reaction.

TABLE 3
Yields of N-methyl-L-valine in the presence of respective strains

| STRAIN | YIELD |
|---|---|
| Mycoplana dimorpha ATCC4279 | 94.8% |
| Mycoplana dimorpha NCIB9439T | 94.0% |

### Comparative Example 1

(1) Culture of pMCA1/JM109 FERM BP-10334

A culture medium having a composition shown in Table 4 was sterilized, and shake cultured at 30° C. for 16 hours after the addition of 50 mg of ampicillin and the inoculation of the genetic recombinant Escherichia coli strain pMCA1/JM109 FERM BP-10334 having hydrolysis activity to an amino acid amide, particularly a sparingly soluble amino acid amide such as phenylglycine amide. The culture fluid obtained was centrifuged to give 1.63 g of the concentrated cell fluid.

TABLE 4
Culture composition (the solution having the following
composition was adjusted to pH 7.0 with a 20% sodium
hydroxide aqueous solution before use)

| | |
|---|---|
| RAW MATERIAL OF CULTURE MEDIUM | USED AMOUNT |

| | | |
|---|---|---|
| Tripton | 10 | g |
| Yeast extract | 5 | g |
| Sodium chloride | 5 | g |
| Water | 100 | ml |

### (2) Hydrolysis of N-Methylvaline Amide

A 100 g amount of a substrate solution was prepared by dissolving 1.0 g of racemic N-methylvaline amide hydrochloride in 50 g of water in a 200 ml flask and further adjusting the solution to pH 8.0 with 20% sodium hydroxide aqueous solution before dilution with water. To the substrate solutions was added 0.5 g of the concentrated cell for fluid obtained in (1) described above, and the mixture was subjected to reaction under stirring with a magnetic stirrer at 35° C. for 1 hour. It was observed in the HPLC measurement of the reaction fluid that none of the hydrolysis of N-methylvaline amide had advanced.

### Comparative Example 2

### (1) Culture of the Other Strains

In the same manner as in pMCA1/JM109 FERM BP-10334 in Comparative Example 1, microorganisms having hydrolysis activity on amino acid amides, Xanthobacter Flavus NCIB10071T, Xanthobacter autotrophicus DSM431 TK0502, Protaminobacter alboflavus NCIB8167, Mycobacterium methanolica BT-84 FERM P-8823, Pseudomonas putida, Rhodococcus erythropolis, Serratia marcescence, and Chromobacterium iodium were inoculated in a culture medium having a composition shown in Table 4 to conduct shake culture at 30° C. for 48 hours. The culture fluid obtained was centrifuged to give a concentrated cell fluid (Table 5).

The results are shown in Table 5.

TABLE 5
Amount of concentrated cell fluid in respective strains

| | YIELD OF |
|---|---|
| STRAIN | CONCENTRATED CELLS |
| Xantobactor Flavus NCIB10071T | 0.97 g |
| Xantobactor autotrophicus DSM431 TK0502 | 1.93 g |
| Protaminobactor alboflavus NCIB8167 | 5.63 g |
| Mycobacterium methanolica BT-84 FERM | 2.00 g |
| P-8823 | |
| Pseudomanas putida | 2.88 g |
| Rhodococcus erythropolis | 3.63 g |
| Serratia marcescence | 4.43 g |
| Chromobacterium iodium | 1.98 g |

### (2) Hydrolysis of N-Methylvaline Amide

A 100 g amount of a substrate solution was prepared by dissolving 1.0 g of racemic N-methylvaline amide hydrochloride in 50 g of water in a 200 ml flask and further adjusting the solution to pH 8.0 with 20% sodium hydroxide aqueous solution before dilution with water. To the respective substrate solutions was added 0.5 g of each of the concentrated cell fluid obtained in (1) described above, and the mixture was subjected to reaction under stirring with a magnetic stirrer at 35° C. for 24 hours. It was observed in the HPLC measurement of the reaction fluid that the hydrolysis of N-methylvaline amide was not advanced with strains except Rhodococcus erythropolis. On the other hand, 57.5% of N-methylvaline amide was hydrolyzed by Rhodococcus erythropolis, but the optical purity of the produced N-methylvaline remained 23% ee due to the excessive amount of the D-form.

### Example 3

The solution after the hydrolysis of N-methylvaline amide in Example 1 was subjected to ultrafiltration with Sartorius Ultrafilter VIVAFL OW200 to remove the cells. To the ultrafiltrate obtained added 1.12 g of 20% sodium hydroxide, and the mixture was concentrated with a rotary evaporator. Furthermore, the concentrated product was vacuum dried at 50° C. The concentrated dry product was diluted with 60 ml of methanol, and the mixture was stirred with a magnetic stirrer at 25° C. for 30 minutes. After stirring, solids were filtered by the suction filtration of the slurry, and then the filtrate was concentrated by a rotary evaporator. The concentrate thus obtained was diluted with 10 ml of acetone, and the mixture was stirred with a magnetic stirrer at 25° C. for 30 minutes. After stirring, the mixture was suction filtered and separated into an acetone solution and solids. The solids were recovered and vacuum dried at 50° C. After drying, N-methyl-L-valine was obtained as a white powder product in a yield of 0.45 g. The recovery of the N-methyl-L-valine based on the N-methyl-L-valine amide in the racemic mixture was 90%. Also, the optical purity of the N-methyl-L-valine obtained was 99% ee.

### Example 4

The acetone solution obtained in Example 3 was concentrated with a rotary evaporator. The concentrate was further dried in vacuum at 50° C. After drying, N-methyl-D-valine amide was obtained as white powder in a yield of 0.45 g.

The recovery of the N-methyl-D-valine amide based on the N-methyl-D-valine amide in the racemic mixture was 90%. In addition, the optical purity of the N-methyl-D-valine amide obtained was 99% ee.

### INDUSTRIAL AVAILABILITY

The present invention allows of economically producing and providing optically active N-methylamino acids and/or optically active N-methylamino acids.

1. A method for preparing an optically active N-methylamino acid, an optically active N-methylamino acid amide, or a mixture thereof, the method comprising:
(A) reacting a cell or a treatment product of a microorganism belonging to the Mycoplana genus with an N-methylamino acid amide of formula (1), to obtain an optically active N-methylamino acid of formula (2) and an optically active N-methylamino acid amide of formula (3) as the enantiomer: 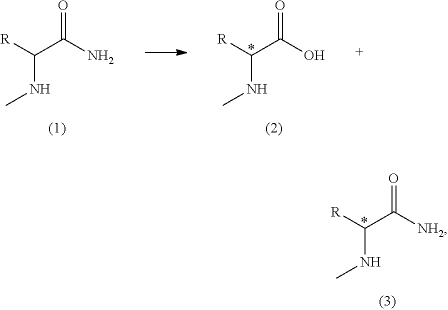
wherein R in formulae (1), (2) and (3) is a linear or branched C1 to C4 alkyl group.

2. The method of claim 1, further comprising:
(B) depositing an optically active amino acid as the crystalline product from a hydrolysis mixture comprising an optically active N-methylamino acid and an optically active N-methylamino acid amide obtained in (A); and then
(C) separating the optically active N-methylamino acid and the optically active N-methylamino acid amide.

3. The method of claim 1, wherein the microorganisms belonging to Mycoplana genus is Mycoplana ramosa or Micoplana dimorpha.

4. The method of claim 1, wherein R in formulae (1), (2), and (3) is an isopropyl group.

5. The method of claim 2, wherein the microorganisms belonging to Mycoplana genus is Mycoplana ramosa or Micoplana dimorpha.

6. The method of claim 2, wherein R in formulae (1), (2), and (3) is an isopropyl group.

7. The method of claim 3, wherein R in formulae (1), (2), and (3) is an isopropyl group.

8. The method of claim 5, wherein R in formulae (1), (2), and (3) is an isopropyl group.

9. The method of claim 1, wherein in formulae (1), (2), and (3) R is a methyl group.

10. The method of claim 1, wherein in formulae (1), (2), and (3) R is an ethyl group.

11. The method of claim 1, wherein in formulae (1), (2), and (3) R is an n-propyl group.

12. The method of claim 1, wherein in formulae (1), (2), and (3) R is an n-butyl group.

13. The method of claim 1, wherein in formulae (1), (2), and (3) R is a sec-butyl group.

14. The method of claim 1, wherein in formulae (1), (2), and (3) R is a tert-butyl group.

15. The method of claim 1, wherein the microorganisms belonging to Mycoplana genus is Micoplana dimorpha.

16. The method of claim 1, wherein the microorganisms belonging to Mycoplana genus is Mycoplana ramosa.
